(19) United States
(12) Patent Application Publication
(10) Pub. No.: US 2011/0130589 A1
(43) Pub. Date: Jun. 2, 2011

(54) PROCESS FOR THE PREPARATION OF BIS(FLUOROALKYL)PHOSPHINIC ACID OR FLUOROALKYLPHOSPHONIC ACID

(75) Inventors: Nikolai Mykola Ignatyev, Duisburg (DE); Dana Bejan, Wuppertal (DE); Helge Willner, Muelheim/Ruhr (DE)

(73) Assignee: Merck Patent Gesellschaft Mit Beschrankter Haftung, Darmstadt (DE)

(21) Appl. No.: 13/055,956

(22) PCT Filed: Jul. 8, 2009
(86) PCT No.: PCT/EP2009/004953

§ 371 Date: Jan. 26, 2011

(30) Foreign Application Priority Data
Jul. 28, 2008 (DE) 102008035174.1

(51) Int. Cl. C07C 68/00; C07F 9/30

(52) U.S. Cl. 560/139; 562/25

(57) ABSTRACT

The invention relates to a process for the preparation of bis(fluoroalkyl)phosphinic acid and/or fluoroalkylphosphonic acid by reaction of, monofluoroalkyltetrafluorophosphorane bis(fluoroalkyl)trifluorophosphorane or tris(fluoroalkyl)difluorophosphorane with water.

The invention relates to a process for the preparation of bis(fluoroalkyl)-phosphinic acid and/or fluoroalkylphosphonic acid by reaction of monofluoroalkyltetrafluorophosphorane, bis(fluoroalkyl)trifluorophosphorane or tris(fluoroalkyl)difluorophosphorane with water.

Bis(fluoroalkyl)phosphinic acids and also fluoroalkylphosphonic acids are known chemicals. Bis(trifluoromethyl)phosphinic acid and trifluoromethylphosphonic acid were prepared by H. J. Emeleus et al., J. Chem. Soc., 1954, 3598-3603 or by H. J. Emeleus et al., J. Chem. Soc., 1955, 563-574. Trifluoromethylphosphonic acid was prepared by oxidative hydrolysis of CF₃PX₂ or (CF₃)₂PX, where X=Cl or I, or by controlled hydrolysis of (CF₃)₃P. However, the synthesis of the starting compounds for these reactions is complex and time-consuming, with the work-up also being included in this 2-step synthesis.

Bis(heptafluoropropyl)phosphinic acid and heptafluoropropylphosphonic acid have been prepared in a similar manner starting from C₃F₇I and red phosphorus in an autoclave at 220 to 230° C., where C₃F₇PI₂ and (C₃F₇)₂PI are formed as intermediates. After separation of (C₃F₇)₂PI, it is reacted further with silver chloride for 11 days to give chlorobisheptafluoropropylphosphine (C₃F₇)₂PCl, which is converted into trichlorobisheptafluoropropylphosphorane (C₃F₇)₂PCl₃. This compound is then finally hydrolysed by reaction with water to give bis(heptafluoropropyl)phosphinic acid. This complex sequence of reactions is known from H. J. Emeleus, J. D. Smith, J. Chem. Soc., 1959, 375-381 and cannot be implemented economically on a large industrial scale.

R. C. Paul, J. Chem. Soc., 1955, 574-575 describes a hydrolysis of tris(trifluoromethyl)phosphine oxide using water to give bis(trifluoromethyl)-phosphinic acid or using sodium hydroxide to give sodium trifluoromethylphosphonate. The disadvantage of this reaction is the difficult access to the starting compound tris(trifluoromethyl)phosphine oxide, which is only possible via tris(trifluoromethyl)dichlorophosphorane, which is accessible with difficulty, by reaction with anhydrous oxalic acid.

Bis(perfluoroalkyl)phosphinic acid can also be prepared by the method of R. P. Singh, J. M. Shreeve, Inorg. Chem., 39, 2000, 1787-1789 by hydrolysis of the corresponding anhydrides, which were in turn prepared by oxidation of bis(perfluoroalkyl)iodophosphine using NO₂.

The reaction of bis(perfluoroalkyl)phosphinyl chlorides with water can be regarded as an alternative to the hydrolysis of bis(perfluoroalkyl)phosphinic anhydride. However, the bis(perfluoroalkyl)phosphinic acid is normally the starting material for the synthesis of bis(perfluoroalkyl)phosphinyl chlorides, meaning that this only represents an academic variant.

WO 2003/087110 discloses a two-step reaction in which firstly tris(perfluoroalkyl)difluorophosphorane is reacted with aqueous HF (hydrofluoric acid), and the tris(perfluoroalkyl)trifluorophosphoric acid H[(perfluoroalkyl)₃-PF₃]*nH₂O formed is hydrolysed further on boiling the aqueous solution.

The conversion of bis(perfluoroalkyl)phosphinic acid into perfluoroalkylphosphonic acid takes place very slowly and requires high temperatures. T. V. Kovaleva et al., J. Gen. Chem. USSR (Engl. trans.), 59, 1989, 2245-2248 describe a hydrolysis of tris(perfluoroalkyl)difluorophosphorane using a 20% sodium hydroxide solution, giving the disodium salt of the perfluoroalkylphosphonic acid, which can be converted into the perfluoroalkylphosphonic acid by treatment with concentrated hydrochloric acid.

Bis(fluoroalkyl)phosphinic acid and fluoroalkylphosphonic acid are, for example, interesting components of proton-conducting membranes or are suitable in accordance with the invention as catalysts in organic chemistry. They are furthermore suitable for use as surfactants per se or for further conversion into the corresponding acid chlorides or acid dichlorides, which are in turn suitable for the synthesis of novel materials, for example ionic liquids. It is therefore desirable to have a synthesis of these compounds available which can be implemented economically on a large industrial scale in order that this interesting class of bis(fluoroalkyl)phosphinic acids or fluoroalkylphosphonic acids can be prepared in large quantities.

The object of the invention is therefore to develop an improved process for the preparation of bis(fluoroalkyl)phosphinic acid and fluoroalkylphosphonic acid or of bis(fluoroalkyl)phosphinic acid or fluoroalkylphosphonic acid which meets the requirements of an economical industrial-scale synthesis.

Surprisingly, it has been found that fluoroalkylfluorophosphoranes can be reacted simply with water by hydrolysis, where the formation of bis(fluoroalkyl)phosphinic acid or fluoroalkylphosphonic acid can be controlled by the reaction temperature and the way in which the reaction is carried out.

The invention therefore relates to a process for the preparation of bis(fluoroalkyl)phosphinic acid and fluoroalkylphosphonic acid or of bis(fluoroalkyl)phosphinic acid or fluoroalkylphosphonic acid by reaction of monofluoroalkyltetrafluorophosphorane, bis(fluoroalkyl)trifluorophosphorane or tris(fluoroalkyl)difluorophosphorane with water.

The starting compounds, i.e. the phosphoranes mentioned, are commercially available or can be prepared as described, for example, in N. Ignat'ev, P. Sartori, J. of Fluorine Chemistry, 103 (2000), p. 57-61; WO 00/21969; U.S. Pat. No. 6,264,818 or WO 98/15562, Merck Patent GmbH, Darmstadt.

In a preferred embodiment, a bis(fluoroalkyl)phosphinic acid and/or fluoroalkylphosphonic acid of the formula I

(CₓF_{2x+1-y}H_{y})ₙP(O)(OH)ₘ I

is prepared, where

x stands for 1, 2, 3, 4, 5, 6, 7, 8, 9, 10, 11 or 12,

y stands for 0, 1, 2, 3, 4 or 5, but where y=0, 1 or 2 for x=1 or 2,

n stands for 1 or 2,

m stands for 1 or 2,

with the proviso that n+m is equal to 3.

x particularly preferably stands for 1, 2, 3 or 4, very particularly preferably for 2 or 4.

y particularly preferably stands for 0.

For the synthesis of these preferred compounds of the formula I, starting compounds of the formula II are employed,

(CₓF_{2x+1-y}H_{y})ₖPFₚ II,

where

x stands for 1, 2, 3, 4, 5, 6, 7, 8, 9, 10, 11 or 12,

y stands for 0, 1, 2 or 3,

k stands for 1, 2 or 3,

p stands for 2, 3 or 4,

with the proviso that k+p is equal to 5.

x particularly preferably stands for 1, 2, 3 or 4, very particularly preferably for 2 or 4.

y particularly preferably stands for 0. In this case, perfluoroalkyl groups, such as, for example, trifluoromethyl, pentafluoroethyl, heptafluoropropyl or linear or branched nonafluorobutyl, are involved. Very particularly preferred perfluoroalkyl groups are pentafluoroethyl and linear nonafluorobutyl.

Mixtures of the bis(fluoroalkyl)phosphinic acid and fluoroalkylphosphonic acid may be formed in the reaction according to the invention. However, the reaction can also be controlled in such a way that only the desired bis(fluoroalkyl)phosphinic acid or the desired fluoroalkylphosphonic acid is formed selectively. This is influenced by the choice of phosphorane as starting material and the temperature programme in the reaction. Reaction with monofluoroalkyltetrafluorophosphorane gives a corresponding fluoroalkylphosphonic acid selectively.

However, it is possible to prepare a fluoroalkylphosphonic acid, for example of the formula I where n=1 and m=2, selectively from a bis(fluoroalkyl)-trifluorophosphorane or tris(fluoroalkyl)difluorophosphorane, for example of the formula II in which k stands for 2 or 3 and p stands for 2 or 3, by mixing the phosphorane with water at temperatures of 50° to 100° C. and carrying out the entire reaction at temperatures of 100° C. to 150° C. The reaction duration for the selective conversion into a corresponding fluoroalkylphosphonic acid is a few days, for example 2 to 14 days.

However, it is also possible to prepare a bis(fluoroalkyl)phosphinic acid, for example of the formula I where n=2 and m=1, selectively from a bis(fluoroalkyl)trifluorophosphorane or tris(fluoroalkyl)difluorophosphorane, for example of the formula II in which k stands for 2 or 3 and p stands for 2 or 3, by mixing the phosphorane with water at temperatures of 0° C. to room temperature for chain lengths of 2 and 3 C atoms in the fluoroalkyl group or mixing the phosphorane with water at temperatures of room temperature to 100° C. for chain lengths from 4 C atoms for the fluoroalkyl group as defined above and carrying out the entire reaction at temperatures of 100° C. to 150° C. The reaction duration for the selective conversion into a corresponding bis(fluoroalkyl)phosphinic acid is a few hours, for example 1 to 24 hours.

The reaction with water takes place without the involvement of a further organic solvent. In general, an excess of water is used, for example a 3-fold to 30-fold excess.

The process according to the invention enables a synthesis of bis(fluoroalkyl)phosphinic acids and/or fluoroalkylphosphonic acids with a yield which is improved compared with the prior art. This process is furthermore suitable for a large-scale industrial synthesis, since water is employed as reagent in the reaction according to the invention and the use of hydrofluoric acid, the handling of which as reagent requires precautionary measures since it is toxic and caustic, is thus unnecessary. The effort involved in separating off the hydrofluoric acid formed in the process is by comparison much less.

The invention furthermore relates to the use of bis(fluoroalkyl)phosphinic acid and/or fluoroalkylphosphonic acid as catalyst in organic chemistry. Bis(fluoroalkyl)phosphinic acid or fluoroalkylphosphonic acid, particularly preferably bis(fluoroalkyl)phosphinic acid, is, for example, a catalyst for
Friedel-Crafts acylations,
Friedel-Crafts alkylations, for example the preparation of 2,6-di-tert-butyl-4-methylphenol,
Friedel-Crafts arylations,
Friedel-Crafts benzylation of aromatic compounds,
condensation of phenols with aromatic aldehydes,
alkylation and oligocondensation of alkanes,
isomerisation and cracking of paraffins (alkanes) or cycloalkanes,
alkylation of aromatic compounds using alcohols, haloalkanes, carbonyl compounds, alkyl esters of carboxylic acids,
isomerisation of alkylbenzene,
cycloalkylation (ring-closure reactions),
carboxylation (Koch-Haat-type reactions) and carbonylation of alkanes and alcohols,
aromatic formylation (Gattermann-Koch-type reactions),
sulfonation and sulfonylation (Friedel-Crafts-type reactions),
nitration,
halogenation (fluorination) of aromatic and aliphatic compounds,
electrophilic amination of aromatic compounds,
oxygenation (hydroxylation) of alkanes, aromatic compounds and natural products (steroids, alkaloids) using ozone (O₃) and hydrogen peroxide or other peroxides,
acylation of alcohols, phenols, thiophenols, aromatic amines,
transformation of carbonyl compounds into O,O-acetals, O,S-acetals or S,S-acetals (protecting-group reactions),
deprotection reactions, for example the conversion of trialkylsilyl ethers into the corresponding alcohols,
synthesis of heterocyclic compounds: oxacycloalkanes and -alkenes, nitrogen-containing heterocyclic compounds, heterocyclic compounds containing two or three heteroatoms,
ring-closure reactions and cycloaddition reactions, for example Diels-Alder reaction, hetero-Diels-Alder reaction, Prins-type reactions,
ring-opening reactions, for example opening of oxiranes,
dehydrogenation of alcohols, diols and polyols for the formation of alkenes, cycloalkenes or ethers,
O-glycosylation of carbohydrates and natural products, such as, for example, glycosyl fluoride,
rearrangement reactions, for example rearrangement of terpenes, Beckmann reaction, Fries, Rupe, Bamberger, Fischer-Hepp, Schmidt and Nazarov reactions,
ionic hydrogenation,
esterification and ester cleavage,
Michael-type additions,
addition of phenols, alcohols, carboxylic acids, sulfonamides, carbamates, acetamides and benzamides onto alkenes,
aminosulfonation of alkenes,
addition of allylsilanes and allylboronates onto aldehydes and cycloalkenes,
Ritter-type reactions,
polymerisation of olefins, ethers and siloxanes,
Mannich- and Aza-Mannich-type reactions,
chemical and electrochemical oxidation,
condensation reactions, for example aldol condensation and dimerisation reaction,
Baeyer-Villiger oxidation,
Thiele-Winter reaction of quinones,
silylation of alcohols,
addition of phosphines onto alkenes,
hydrogenation of alkynes,
synthesis of sulfonium and selenium salts.

The bis(fluoroalkyl)phosphinic acid or fluoroalkylphosphonic acid is particularly preferably employed in accordance with the invention in Friedel-Crafts-type reactions as described above, very particularly preferably in Friedel-Crafts acylations.

The corresponding bis(fluoroalkyl)phosphinic acid or fluoroalkylphosphonic acid is generally used in an amount of 0.01 to 15 mol %, based on the employed amount of the compound to be acylated. 1 mol % to 10 mol % of catalyst are particularly preferably used. The other reaction conditions are not modified and are known to the person skilled in the art from the specialist literature. Examples 5 and 6 confirm the use according to the invention. The use of the catalysts according to the invention produces an excellent increase in yield. This is documented by Comparative Example 7.

The corresponding bis(fluoroalkyl)phosphinic acid or fluoroalkylphosphonic acid can also be used in a mixture with other Brønsted or Lewis acids.

It is known from the literature [G. A. Olah, G. K. Surya Prakash, A. Molnar, J. Sommer, Superacid Chemistry, 2nd Ed., Wiley, 2009, 501-788] that the reactions described above can be catalysed by perfluoroalkanesulfonic acids, for example trifluoromethanesulfonic acid. These strong acids are effective catalysts (Example 8). However, known catalysts of this type are persistent owing to the high hydrolytic stability. It is also known, for example, that the production of perfluorooctanesulfonic acid has already been restricted in the USA for these reasons.

Bis(perfluoroalkyl)phosphinic acid and fluoroalkylphosphonic acid are less stable to hydrolysis, in particular in basic media. Hydrolysis of these compounds produces phosphates and volatile monohydroperfluoroalkanes, for example C₂F₅H (F 125), which are employed as non-toxic ozone-friendly substitutes for chlorofluorocarbons.

Even without further comments, it is assumed that a person skilled in the art will be able to utilise the above description in the broadest scope. The preferred embodiments and examples should therefore merely be regarded as descriptive disclosure which is absolutely not limiting in any way.

### EXAMPLES

The NMR spectra were measured on solutions in deuterated solvents at 20° C. in a Bruker Avance 400 spectrometer with a 5 mm ¹H/BB broadband probe with deuterium lock, unless indicated in the examples. The measurement frequencies of the various nuclei are: ¹H, 400.13 MHz, ¹⁹F: 376.49 MHz and ³¹P: 161.97 MHz. The referencing method is indicated separately for each spectrum or each data set.

### Example 1

### Synthesis of Bis(Pentafluoroethyl)Phosphinic Acid, (C₂F₅)₂P(O)OH

250 g (0.59 mol) of tris(pentafluoroethyl)difluorophosphorane are slowly added at room temperature with vigorous stirring to 86 g (4.78 mol) of water. The solution obtained is heated at a temperature of 100° to 110° C. (oil-bath temperature 120° C.) for 24 hours. The completeness of the reaction is monitored by ³¹P-NMR measurements. The aqueous HF is then distilled off. The residue is subsequently distilled under reduced pressure, giving 134.4 g of liquid bis(pentafluoroethyl)phosphinic acid, which corresponds to a yield of 76%, based on tris(pentafluoroethyl)difluorophosphorane.

Boiling point: 54-56° C. (0.6 mbar) (literature: 63-64° C. (125 Pa) from WO 03/087110).

The NMR spectra are measured for the pure substance with an acetonitrile-d3 film as external lock.

¹H NMR (reference TMS), δ, ppm: 12.6 s (OH).

¹⁹F NMR (reference CCl₃F), δ, ppm: −81.9 m (2CF₃); −127.0 d (2CF₂); ²J_{P,F}=85 Hz.

³¹P NMR (reference 85% H₃PO₄, solvent CD₃CN), δ, ppm: 6.7 quin; ²J_{P,F}=84 Hz.

### Example 2

### Synthesis of bis(nonafluorobutyl)phosphinic acid, (C₄F₉)₂P(O)OH

117 g (0.16 mol) of tris(nonafluorobutyl)difluorophosphorane are slowly added with vigorous stirring to 55 g (3.06 mol) of water, where the water has a temperature of between 90° and 100° C. The solution obtained is heated under reflux (temperature 100-125° C.) for 1 hour. The completeness of the reaction is monitored by ³¹P-NMR measurements. The aqueous HF is then distilled off. The residue is subsequently dried under reduced pressure at an oil-bath temperature of 50° C., giving 75.8 g of solid bis(nonafluorobutyl)phosphinic acid, which corresponds to a yield of 94%, based on tris(nonafluorobutyl)difluorophosphorane.

¹H NMR (reference TMS, solvent CD₃CN), δ, ppm: 12.1 s (OH).

¹⁹F NMR (reference CCl₃F, solvent CD₃CN), δ, ppm: −82.4 μm (2CF₃); −121.8 br.s (2CF₂); −122.4 d (2CF₂); −127.0 m (2CF₂); ²J_{P,F}=85 Hz; ⁴J_{F,F}=10 Hz.

³¹P NMR (reference 85% H₃PO₄, solvent CD₃CN), δ, ppm: 3.9 quin; ²J_{P,F}=85 Hz.

### Example 3

### Synthesis of Pentafluoroethylphosphonic Acid, (C₂F₅)P(O)(OH)₂

222 g (0.52 mol) of tris(pentafluoroethyl)difluorophosphorane are slowly added with vigorous stirring to 120 g (6.67 mol) of water, where the water has a temperature of between 95° and 100° C. The solution obtained is heated under reflux (oil-bath temperature 100-125° C.) for 14 days. The completeness of the reaction is monitored by ³¹P-NMR measurements. Aqueous HF is then distilled off. The residue is subsequently dried under reduced pressure at an oil-bath temperature of 60-70° C. for 10 hours, giving 103 g of liquid pentafluoroethylphosphonic acid, which corresponds to a yield of 99%, based on tris(pentafluoroethyl)difluorophosphorane.

The NMR spectra are measured for the pure substance with an acetonitrile-d3 film as external lock.

¹H NMR (standard: TMS), δ, ppm: 11.3 s (20H).

¹⁹F NMR (standard: CCl₃F), δ, ppm: −82.7 br.s (CF₃), −128.4 d (CF₂), ²J_{P,F}=89 Hz.

³¹P NMR (standard: 85% H₃PO₄), δ, ppm: −1.3 t, ²J_{P,F}=89 Hz.

### Example 4

### Synthesis of N-Nonafluorobutylphosphonic Acid, (C₄F₉)P(O)(OH)₂

115 g (0.16 mol) of tris(nonafluorobutyl)difluorophosphorane are slowly added with vigorous stirring to 60 g (3.33 mol) of water, where the water has a temperature of between 90° and 96° C. The solution obtained is heated under reflux (temperature 100-125° C.) for 4 days. The completeness of the reaction is monitored by ³¹P-NMR measurements. Aqueous HF is then distilled off. The residue is subsequently dried under reduced pressure at an oil-bath temperature of 60-70° C., giving 47 g of solid n-nonafluorobutylphosphonic acid, which corresponds to a yield of 99%, based on tris(nonafluorobutyl)difluorophosphorane.

¹H NMR (solvent: CD₃CN; standard: TMS), δ, ppm: 11.1 s (20H).

¹⁹F NMR (solvent: CD₃CN; standard: CCl₃F), δ, ppm: −82.4 μm (CF₃), −122.5 m (CF₂), −124.8 d, t, m (CF₂), −127.1 μm (CF₂), ²J_{P,F}=88 Hz, ⁴J_{F,F}=14 Hz, ⁴J_{F,F}=11 Hz.

³¹P NMR (solvent CD₃CN; standard: 85% H₃PO₄), δ, ppm: −1.1 t, ²J_{P,F}=90 Hz.

### Example 5

### Acylation of β-Naphthol Using Acetic Anhydride in the Presence of (C₂F₅)₂P(O)OH

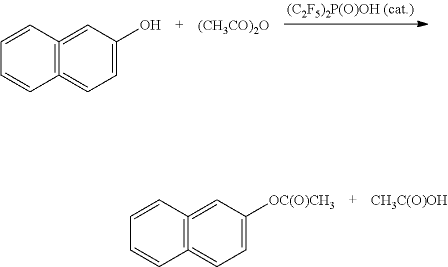

a) 0.845 g (8.28 mmol) of acetic anhydride (Ac₂O) is added at room temperature to a mixture of 1 g (6.9 mmol) of β-naphthol and 0.02 g (0.06 mmol; 1 mol %) of (C₂F₅)₂P(O)OH in 2.5 cm³ of CH₂Cl₂. The reaction mixture is stirred at room temperature for a further 30 minutes. The solution obtained is subsequently concentrated under reduced pressure, and the residue is taken up in diethyl ether (Et₂O, 30 cm³). The organic phase is washed with water and 2% aqueous NaOH and saturated NaCl solution. Drying over MgSO₄ and distillation of the diethyl ether under reduced pressure gives 1.24 g (6.6 mmol) of 2-acetoxynaphthalene as a solid. The yield of 2-acetoxynaphthalene is 96%, based on the amount of β-naphthol employed.

The measured melting point of 70° C. corresponds to the literature value [P. Baumgarten, Berichte der Deutschen Chemischen Gesellschaft, 60B (1927), pp. 1174-1178; A. McKillop, E. C. Taylor, Ger. Offen. DE 69-1903598, US 68-700352]. ¹H- and ¹³C-NMR spectra likewise correspond to the literature data [P. Granger and M. Maugras, Organic Magnetic Resonance, 1975, Vol. 7, pp. 598-601].

b) A mixture of 0.246 g (1.7 mmol) of β-naphthol, 0.209 g (2.04 mmol) of Ac₂O and 0.05 g (0.165 mmol; 10 mol %) of (C₂F₅)₂P(O)OH is stirred at room temperature for 30 minutes. A solid is formed, which is washed with water and extracted with diethyl ether. The organic phase is subsequently washed with 2% aqueous NaOH and saturated NaCl solution. Drying using MgSO₄ and removal of the diethyl ether by distillation under reduced pressure gives 0.3 g (1.6 mmol) of 2-acetoxynaphthalene, which corresponds to a yield of 94%, based on the amount of β-naphthol employed. The melting point and NMR spectra correspond to the values measured for the product from Example 5a.

### Example 6

### Acylation of β-Naphthol Using Acetic Anhydride in the Presence of (C₄F₉)₂P(O)OH

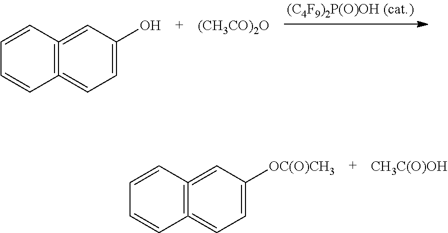

0.42 g (4.11 mmol) of acetic anhydride (Ac₂O) is added at room temperature to a mixture of 0.5 g (3.46 mmol) of β-naphthol and 0.017 g (0.03 mmol; 1 mol %) of (C₄F₉)₂P(O)OH in 3 cm³ of CH₂Cl₂. The reaction mixture is stirred at room temperature for a further 30 minutes. The solution obtained is subsequently concentrated under reduced pressure, and the residue is taken up in diethyl ether (Et₂O). The organic phase is washed with water and 2% aqueous NaOH and saturated NaCl solution. Drying over MgSO₄ and distillation of the diethyl ether under reduced pressure gives 0.62 g (3.3 mmol) of 2-acetoxynaphthalene as a solid. The yield of 2-acetoxynaphthalene is 96%, based on the amount of β-naphthol employed.

The melting point and NMR spectra correspond to the values measured for the product from Example 5a.

### Example 7

### Acylation of β-Naphthol Using Acetic Anhydride in the Absence of a Catalyst According to the Invention

0.86 g (8.4 mmol) of acetic anhydride (Ac₂O) is added at room temperature to a mixture of 1 g (6.9 mmol) of β-naphthol in 2.5 cm³ of CH₂Cl₂. The reaction mixture is stirred at room temperature for a further 30 minutes. The solution obtained is subsequently concentrated under reduced pressure, and the residue is taken up in diethyl ether (Et₂O). The organic phase is washed with water and 2% aqueous NaOH and saturated NaCl solution. Drying over MgSO₄ and distillation of the diethyl ether under reduced pressure gives 0.13 g (0.7 mmol) of 2-acetoxynaphthalene as a solid. The yield of 2-acetoxynaphthalene is 10%, based on the amount of β-naphthol employed.

The product obtained has a melting point of 65° C., which is 5° C. lower compared with the 2-acetoxynaphthalene from Example 5, which indicates impurities in the product.

### Example 8

### Acylation of β-Naphthol Using Ac₂O in the Presence of Trifluoromethanesulfonic Acid, CF₃SO₃H

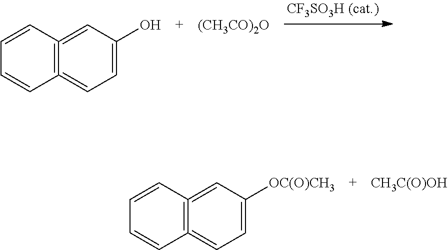

0.02 g (0.13 mmol; 1.9 mol %) of CF₃SO₃H is added at room temperature to a mixture of β-naphthol (1.01 g, 7.0 mmol) and acetic anhydride, Ac₂O (0.856 g, 8.4 mmol) in CH₂Cl₂ (2.5 cm³). The reaction mixture is stirred for a further 30 minutes. The homogeneous solution obtained is concentrated under reduced pressure. The solid residue obtained is taken up in diethyl ether. The organic phases are washed with water, 2% aqueous NaOH and saturated sodium chloride solution. Drying over MgSO₄ and removal of the organic solvent under reduced pressure gives 1.25 g (6.7 mmol) of a white solid. The yield of 2-acetoxynaphthalene is 95.7%, based on the amount of β-naphthol employed. The melting point and NMR spectra are identical to the values of Example 5a.

1. Process for the preparation of bis(fluoroalkyl)phosphinic acid and fluoroalkylphosphonic acid or of bis(fluoroalkyl)phosphinic acid or fluoroalkylphosphonic acid by reaction of monofluoroalkyltetrafluorophosphorane, bis(fluoroalkyl)trifluorophosphorane or tris(fluoroalkyl)difluorophosphorane with water.

2. Process according to claim 1, characterised in that a bis(fluoroalkyl)phosphinic acid and/or fluoroalkylphosphonic acid of the formula I
(CₓF_{2x+1-y}H_{y})ₙP(O)(OH)ₘ I
is prepared, where
x stands for 1, 2, 3, 4, 5, 6, 7, 8, 9, 10, 11 or 12,
y stands for 0, 1, 2, 3, 4 or 5, but where y=0, 1 or 2 for x=1 or 2,
n stands for 1 or 2,
m stands for 1 or 2,
with the proviso that n+m is equal to 3.

3. Process according to claim 1, characterised in that the phosphorane employed is a compound of the formula II
(CₓF_{2x+1-y}H_{y})ₖPFₚ II
where
x stands for 1, 2, 3, 4, 5, 6, 7, 8, 9, 10, 11 or 12,
y stands for 0, 1, 2 or 3,
k stands for 1, 2 or 3,
p stands for 2, 3 or 4,
with the proviso that k+p is equal to 5.

4. Process according to claim 1, characterised in that the reaction is carried out at temperatures of 100° C. to 150° C.

5. A method of catalyzing an organic reaction comprising employing as catalyst a bis(fluoroalkyl)phosphinic acid and/or fluoroalkylphosphonic acid.
